(19) United States
(12) Patent Application Publication
(10) Pub. No.: US 2025/0352559 A1
(43) Pub. Date: Nov. 20, 2025

(54) Transdermal Delivery Composition for Delivery of at least one Glucose Controlling Agent, and Method of Delivering at least one Glucose Controlling Agent

(71) Applicant: Skinvisible Pharmaceuticals, Inc., Las Vegas, NV (US)

(72) Inventors: James Roszell, Las Vegas, NV (US); Terry Howlett, Las Vegas, NV (US)

(21) Appl. No.: 19/208,087

(22) Filed: May 14, 2025

Related U.S. Application Data
(60) Provisional application No. 63/647,993, filed on May 15, 2024.
(60) Provisional application No. 63/661,428, filed on Jun. 18, 2024.

(51) Int. Cl. A61K 31/00; A61K 31/05; A61K 31/357; A61K 31/4196; A61K 31/454; A61K 31/7048; A61K 38/26; A61K 45/00

(52) U.S. Cl. CPC A61K 31/658; A61K 31/05; A61K 31/357; A61K 31/4196; A61K 31/454; A61K 31/7048; A61K 38/26; A61K 45/00

(57) ABSTRACT

A transdermal delivery composition including: a therapeutic effective amount of at least one glucose controlling agent; a polymer component in an amount sufficient to hold the at least one glucose controlling agent and provide a sustained release of the at least one glucose controlling agent from the transdermal composition after application to skin tissue; a penetrant component in an amount sufficient to assist with a transdermal penetration of the at least one glucose controlling agent; a surfactant component in an amount sufficient to stabilize the polymer adduct and release the at least one glucose controlling agent from the transdermal delivery composition after application to skin tissue; and water. Methods of delivery are provided.

### RELATED APPLICATIONS

This application claims priority, to the extent appropriate, to U.S. provisional patent application Ser. No. 63/661,428, filed Jun. 18, 2024, and to U.S. provisional patent application Ser. No. 63/647,993, filed May 15, 2024. The entire disclosures of these application are incorporated by reference herein.

### BACKGROUND

Obesity and metabolic disorders, such as, type 2 diabetes and dyslipidemia, are prevalent and complex medical conditions with significant health implications. Despite extensive research efforts, obesity stands as a formidable global health challenge, precipitating a multitude of detrimental health outcomes such as cardiovascular disease, type 2 diabetes, certain cancers, and a myriad of other metabolic complications. Despite considerable advancements in pharmacotherapy, the landscape of safe and effective treatment options remains limited, highlighting the pressing need for innovative therapeutic approaches.

Modulation of the endocannabinoid system through CB1 receptor antagonism and the augmentation of GLP-1 receptor activity have emerged as promising therapeutic strategies in the realm of obesity management. However, the translation of these promising avenues into clinical practice has been impeded by challenges in drug delivery.

The endocannabinoid system and the glucagon-like peptide-1 (GLP-1) pathways are targets for therapies targeting obesity and metabolic disorders. CB1 receptor antagonists have demonstrated significant weight losses by modulating the endocannabinoid system and reducing food intake and adipogenesis. Similarly, GLP-1 receptor agonists have shown promising results in promoting weight loss, improving glycemic control, and reducing cardiovascular risk factors.

However, clinical translation of these promising therapeutic agents has been hindered by challenges associated with conventional administration routes (pills or injection), such as poor bioavailability, variable absorption rates, and systemic side effects. For example, certain agents have low bioavailability as pills due to degradation in the stomach and intestines and also by the challenge of crossing the intestinal epithelium and into the bloodstream. The presence of food in the digestive tract can also affect absorption and release.

Transdermal drug delivery systems offer compelling alternatives, enabling targeted and sustained delivery of therapeutic agents directly to adipose tissue and target sites while minimizing systemic exposure and associated side effects.

### SUMMARY

A transdermal delivery composition for the delivery to skin and the penetration of the skin is provided by the present disclosure. The transdermal delivery composition includes: (a) a therapeutic effective amount of at least one glucose controlling agent; (b) a polymer component in an amount sufficient to hold the at least one glucose controlling agent and provide a sustained release of the at least one glucose controlling agent; (c) a penetrant component in an amount sufficient to assist with a transdermal penetration of the at least one glucose controlling agent through the skin tissue after the composition has been applied to the skin tissue; (d) a surfactant component in an amount sufficient to stabilize the polymer adduct and release the at least one glucose controlling agent from the transdermal delivery composition after application to the skin tissue; and (e) water.

A glucose controlling agent includes an agent that effects the blood sugar level and preferably adjusts the blood sugar level downwardly. Exemplary glucose controlling agents include, for example, a CB-1 receptor antagonist, a GLP-1 receptor agonist, a SGLT-2 receptor antagonist, or a combination thereof.

A transdermal delivery composition for the delivery to skin and the penetration of the skin is provided by the present disclosure. The transdermal delivery composition includes: (a) a therapeutic effective amount of a CB-1 receptor antagonist comprising olivetol, LH-21, rimonabant, taranabant, tetrahydrocannabivarin, or mixtures thereof; (b) a polymer component in an amount sufficient to hold the CB-1 receptor antagonist and provide a sustained release of the CB-1 receptor antagonist after application to the skin tissue; (c) a penetrant component in an amount sufficient to assist with a transdermal penetration of the CB-1 receptor antagonist through the skin tissue after the composition has been applied to the skin tissue; (d) a surfactant component in an amount sufficient to stabilize the polymer adduct and release the CB-1 receptor antagonist mixture from the transdermal delivery composition upon application to the skin tissue; and (e) water.

A method of application or delivery of a transdermal delivery composition to skin tissue is provided by the present disclosure. The method includes applying the transdermal delivery composition to the skin tissue and spreading the composition on the skin tissue.

### BRIEF DESCRIPTION OF THE DRAWINGS

FIG. 1 is a graph showing the release and membrane penetration of Olivetol released over time at a dose of 6.77 mg/g during a Franz Cell study.

FIG. 2 is a graph showing the release and membrane penetration of LH-21 released over time at a dose of 4.42 mg/g during a Franz Cell study.

FIG. 3 is a graph showing the release and membrane penetration of Danuglipron released over time during a Franz Cell study.

FIG. 4 is a graph showing the release and membrane penetration of Tetrahydrocannbivarin (THC-V) over time during a Franz Cell study.

### DETAILED DESCRIPTION

A composition for transdermal delivery of at least one glucose controlling agent through skin tissue can be referred to as a transdermal delivery composition or more conveniently referred to as a delivery composition or as a composition. The glucose controlling agent can include at least one CB-1 receptor antagonist, at least one GLP-1 receptor agonist, at least one SGLT-2 receptor antagonist, or a combination of any two or more of the at least one CB-1 receptor antagonist, the at least one GLP-1 receptor agonist, or the at least one SGLT-2 receptor antagonist. The CB-1 receptor antagonists and/or GLP-1 receptor agonists and/or SGLT-2 receptor antagonist can be referred to as active ingredient(s) or more conveniently as active(s). Herein, the reference to actives is meant to include CB-1 receptor antagonists, GLP-1 receptor agonists, and SGLT-2 receptor antagonists that would be efficacious in delivery through skin tissue as a result of application in a transdermal delivery composition according to the present disclosure. CB-1 receptor antagonists are generally considered to act by blocking the activation of CB-1 receptors, GLP-1 receptor agonists are generally considered to act by binding to GLP-1 receptors, and SGLT-2 receptor antagonists are generally considered to act by blocking activation of SGLT-2 receptors. It should be appreciated that the composition for transdermal delivery can include any combination of CB-1 receptor antagonists, GLP-1 receptor agonists, and SGLT-2 receptor antagonists, and it may be expected that this composition may provide the most desired results for, for example, weight loss. Also, it should be appreciated that the composition can include CB-1 receptor antagonists and no GLP-1 receptor agonists and no SGLT-2 receptor antagonists, can include GLP-1 receptor agonists and no CB-1 receptor antagonists and no SGLT-2 receptor antagonists, or can include SGLT-2 receptor antagonists and no CB-1 receptor antagonists and no GLP-1 receptor agonists. Furthermore, the composition can include single or multiple of the CB-1 receptor antagonists, the composition can include single or multiple of the GLP-1 receptor agonists, and the composition can include single or multiple of the SGLT-2 receptor antagonists. The use of “(s)” following active ingredient or active should be understood to mean that a single active or multiple different actives can be provided.

The reference to a glucose controlling agent includes an agent that effects the blood sugar level and preferably adjusts the level downwardly. Exemplary glucose controlling agents include, for example, a CB-1 receptor antagonist, a GLP-1 receptor agonist, a SGLT-2 receptor antagonist, or a combination thereof. It should be appreciated that the glucose controlling agent is preferably not a sugar or a sugar component. Furthermore, by controlling the blood sugar level, the glucose controlling agent can be considered to effect blood sugar levels and would be considered for weight loss and or controlling diabetes.

The purpose of the transdermal delivery composition is to deliver at least one active to skin tissue over time, and permits the at least one active to be taken systemically through the skin tissue. As a result, the transdermal delivery composition is provided so that it holds onto the at least one active while also releasing the at least one active over a time period of about 4 to 12 hours, and preferably about 4 to 6 hours. During this time period, the release preference is a release that can be considered relatively consistent and sustained. This release can also be referred to as a prolonged release since a majority of the at least one active is not released within the first half hour. Accordingly, the transdermal delivery composition provides for both sustained and prolonged release, and also enhanced transmission or uptake through the skin tissue. The transdermal delivery composition can provide a sustained release and permeation of the at least one active over a period of at least four hours, and preferably six hours, wherein the release in each hour after the first hour can be a release and permeation of the active that is between about 50% and 200% of the release and permeation of the active in a preceding hour and is based on a Franz Cell Study. Preferably, the release and permeation of the active is between about 75% and 150% of the release and permeation of the active in a preceding hour.

The transdermal delivery composition can include a polymer component that holds onto the at least one active and releases the at least on active over time after application to skin tissue. The polymer component can be a polymer adduct which can be considered a hydrophilic polymer/hydrophobic polymer adduct that holds onto the at least one active and provide a desired release of the at least one active over time. In addition, the transdermal delivery composition can include surfactants for helping solubilize the polymer component, such as the hydrophobic polymers/hydrophilic polymer adduct, and for assisting in the release of the at least one active therefrom, for example, from the polymer adduct. In addition, the transdermal delivery composition can include a penetrant component to assist with the penetration of the at least one active through the skin tissue. The transdermal delivery composition can additionally include emollients, chelating agents, antioxidants, preservatives, and a pH neutralizer.

The transdermal delivery composition can be summarized Table 1 below.

TABLE 1
Transdermal Delivery Composition

| Component | Amount (wt. %) |
|---|---|
| Water | 70-90, preferred 75-85 |
| Polymer component (such as | 4-9, preferred 4.5-8, more |
| polymer adduct) | preferred 5-6 |
| Surfactants | 3-9, preferred 3.5-8, more |
| | preferred 4-6 |
| Penetrants | 2-10, preferred 3-7, more |
| | preferred 3.5-5 |
| CB-1 receptor antagonists or GLP- | 0.05-4, preferred 0.1-3, more |
| 1 receptor agonists or SGLT-2 | preferred 0.2-2 |
| receptor antagonist or combination | |
| thereof | |
| Antioxidants | Optional, but if present, |
| | present in a range of 0.1-1, |
| | preferred 0.2-0.5 |
| Chelating agent | Optional, but if present, |
| | present in a range of 0.01-0.1 |
| Preservative | Optional, but if present, |
| | present in a range of 0.1-2, |
| | preferably 0.5-1.5 |
| Emollient | Optional, but if present, |
| | present in a range of 1-5, |
| | preferably 1.5-4 |
| pH neutralizing agent | Amount affective to provide |
| | a pH of 6-7.5 |

### CB-1 Receptor Antagonists

CB1 receptors are expressed in various tissues, including the brain, adipose tissue, liver, and skeletal muscle, and play a crucial role in regulating energy homeostasis, lipid metabolism, and appetite (Pagotto et al., 2006; Silvestri and Di Marzo, 2013). The following described CB-1 receptor antagonists can be used in the claimed transdermal delivery composition.

CB1 receptor antagonists, such as olivetol, LH21, rimonabant, and taranabant, have been shown to block the activation of CB1 receptors, leading to a reduction in food intake, increased energy expenditure, and improved metabolic parameters (Rumberger et al., 2014; Jiang et al., 2018; Pagotto et al., 2006; Silvestri and Di Marzo, 2013). LH21 is generally known as a peripheral cannabinoid receptor 1 antagonist. Additional CB1 receptor antagonists include Monlunabant, INV-347, and INV-202 (CagriSema) (available from Novo Nordisk).

In in vitro studies, olivetol and LH21 have been demonstrated to inhibit adipocyte differentiation and lipid accumulation in 3T3-LI preadipocytes, suggesting their potential in modulating adipogenesis (Rumberger et al., 2014; Jiang et al., 2018). In addition, treatment with CB1 antagonists has been found to upregulate the expression of lipolytic genes and enzymes, such as hormone-sensitive lipase (HSL) and adipose triglyceride lipase (ATGL), in adipocytes (Rumberger et al., 2014; Jiang et al., 2018; Pagotto et al., 2006).

In in vivo studies, administration of olivetol, LH21, rimonabant, and taranabant has been shown to reduce body weight gain, decrease adipose tissue mass, and improve glucose tolerance and insulin sensitivity in animal models of diet-induced obesity (Rumberger et al., 2014; Jiang et al., 2018; Pagotto et al., 2006; Silvestri and Di Marzo, 2013). In addition, chronic treatment with certain CB1 antagonists has been demonstrated to ameliorate obesity-associated metabolic abnormalities, such as hyperglycemia, dyslipidemia, and hepatic steatosis (fatty liver), in obese rodents (Rumberger et al., 2014; Jiang et al., 2018; Pagotto et al., 2006; Silvestri and Di Marzo, 2013).

In clinical studies, rimonabant (Acomplia®) was the first CB1 antagonist approved for the treatment of obesity and related metabolic disorders in several countries, including the European Union. However, it was later withdrawn from the market due to safety concerns, including an increased risk of psychiatric adverse effects (Silvestri and Di Marzo, 2013; Topol et al., 2010). Taranabant, another CB1 antagonist, has been evaluated in clinical trials for the treatment of obesity and showed promising results in reducing body weight and improving metabolic parameters. However, its development was discontinued due to potential safety concerns (Silvestri and Di Marzo, 2013; Addy et al., 2008).

Regarding safety and bioavailability, preclinical studies have suggested that olivetol and LH21 have favorable safety profiles and are well-tolerated at therapeutic doses (Rumberger et al., 2014; Jiang et al., 2018). Investigations into the pharmacokinetics and bioavailability of olivetol, LH-21, rimonabant, and taranabant have been conducted, providing insights into their absorption, distribution, metabolism, and excretion (Jiang et al., 2018; Silvestri and Di Marzo, 2013). These findings support the potential use of CB1 antagonists, such as olivetol, LH21, rimonabant, and taranabant, as therapeutic agents for the treatment of obesity and related metabolic disorders. However, it is important to consider the potential safety concerns associated with some of these compounds, particularly in light of the withdrawal of rimonabant from the market. Additional preclinical and clinical studies may be required to further establish the safety, efficacy, and optimal dosing regimens of these CB1 antagonists before they can be considered for commercial development and clinical use.

Another CB1 receptor antagonist that may be used includes Tetrahydrocannabivarin (THCV) which is part of the natural endocannabinoid system. THCV has been shown to have many favorable biological effects including appetite suppression, lowering of blood pressure, A1C, and insulin resistance.

### GLP-1 Receptor Agonists

Alternatively, the system may incorporate a therapeutically effective amount of a GLP-1 receptor agonist. The following described GLP-1 receptor agonists can be used in the claimed transdermal delivery composition. Exemplary GLP-1 receptor agonists include exenatide, liraglutide, Orforglipron, Lotiglipron, Danuglipron, GT-001, GDD-3898, GSBR-1290, RG 6652 (CT-996), or structurally related compounds with suitable physicochemical properties for transdermal delivery. Glucagon-like peptide-1 (GLP-1) receptor agonists are another class of drugs that have shown promise in the management of obesity and metabolic disorders. These drugs act by increasing satiety, slowing gastric emptying, and improving glucose homeostasis, leading to weight loss and improved glycemic control. For purposes of this disclosure, GLP-1 receptor agonists include those agonists that are often referred to as GLP-1RA.

Liraglutide, a once-daily GLP-1 receptor agonist, has been extensively studied for its weight loss effects. In a randomized, double-blind, placebo-controlled trial (SCALE Obesity and Prediabetes) involving 3,731 overweight or obese patients without type 2 diabetes, treatment with liraglutide (3.0 mg/day) for one year resulted in significant weight loss compared to placebo (mean weight loss of 8.0 kg vs. 2.6 kg, respectively) (Pi-Sunyer et al., 2015). Additionally, liraglutide improved several metabolic parameters, including waist circumference, blood pressure, and lipid profiles.

Exenatide, a twice-daily GLP-1 receptor agonist, has also been evaluated for its anti-obesity effects. In a randomized, double-blind, placebo-controlled trial involving 216 overweight or obese patients, treatment with exenatide (10 μg twice daily) for 24 weeks resulted in significant weight loss compared to placebo (mean weight loss of 5.1 kg vs. 1.6 kg, respectively) (Astrup et al., 2009). Exenatide also improved several metabolic parameters, including waist circumference, blood pressure, and lipid profiles.

The GLP-1 receptor agonists can include dual GLP-1/GIP receptor agonists. Such dual GLP-1/GIP receptor agonists include RG6641 (CT-868) and RG 6640 (CT-388) (Roche). The dual GLP-1/GIP receptor agonists are believed to function by increasing the body's sensitivity to insulin by lowering blood sugar thereby releasing more insulin in combination with lowering sugar production by the liver and slowing digestion.

### SGLT-2 Receptor Antagonists

Alternatively, the system may incorporate a therapeutically effective amount of a SGLT-2 receptor antagonist. An SGLT-2 receptor antagonist is generally understood as a sodium-glucose co-transporter 2 inhibitor. The following described SGLT-2 receptor antagonists can be used in the claimed transdermal delivery composition. Examples include canagliflozin (Invokana) (Janssen Pharma), ertugliflozin (Steglatro) (Pfizer), dapagliflozin (Farxiga) (AstraZeneca & Bristol-Myers Squibb), and empagliflozin (Jardiance) (Boehringer Ingelheim). It is understood that the SGLT-2 receptor antagonists act to reduce body weight by removing glucose in the kidneys. SGLT2 inhibit the sodium-glucose co-transporter 2 (SGLT2) protein, which is responsible for reabsorbing glucose from the kidney's filtrate back into the bloodstream.

Clinical trial data demonstrates the potential of CB1 receptor antagonists and GLP-1 receptor agonists as effective pharmacological interventions for the management of obesity and associated metabolic complications. Transdermal delivery of these agents offer advantages over traditional oral or injectable formulations, such as elimination of potentially toxic first pass through the liver, improved patient compliance, sustained drug release, and reduced systemic side effects.

In general, the active ingredient(s) can have any size with the proviso that it can penetrate or pass through the skin tissue. If the active ingredient(s) are too large, it may be difficult for the active ingredient(s) to pass through the skin. Accordingly, a maximum molar mass of active ingredient(s) to penetrate the skin can be about 1000 g/mole or Da. If the active ingredient(s) are close to about 1000 g/mole, it may be helpful for the active ingredient(s) to have a neutral charge and have lipophilic properties. In addition, the molar mass of the active ingredient(s) may be less than about 1000 g/mole and alternatively or preferably less than about 600 g/mole to provide desired penetration through the skin, and alternatively or preferably can have a molar mass of about 400 g/mole or less. The molecule size is a significant drawback for transdermal application because many of CB-1 receptor antagonists and GLP-1 receptor agonists that are known and used for oral or intravenous applications are proteins or peptides that are too large to successfully pass through skin tissue. For example, semaglutide (Ozempic) has a molar mass of 4113.64 g/mol which is far too large to pass through the skin. Likewise, liraglutide and exenatide are too large to penetrate the skin.

Olivetol has the following structure and a molar mass of 180.25 g/mol.

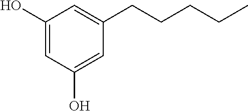

LH-21 has the following structure and a molar mass of 408.8 g/mole.

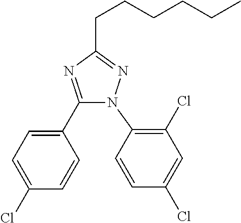 [SMILES: CCCCCCc1nc(-c2ccc(Cl)cc2)n(-c2ccc(Cl)cc2Cl)n1]

Danuglipron has the following structure and a molar mass of 555.61 g/mol.

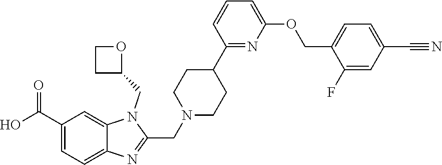 [SMILES: N#Cc1ccc(COc2cccc(C3CCN(Cc4nc5ccc(C(=O)O)cc5n4C[C@@H]4CCO4)CC3)n2)c(F)c1]

Tetrahydrocannabivarin (THCV) has the following structure and a molar mass of 286.4 g/mole.

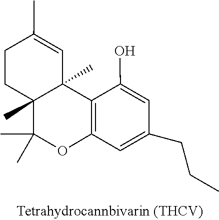 [SMILES: CCCc1cc(O)c2c(c1)OC(C)(C)[C@]1(C)CCC(C)=C[C@@]21C]

In general, a transdermal delivery process of delivering a topical product through skin tissue involves a two-step process. First, the active ingredient(s) are released from the vehicle, in this case the polymer component. Active ingredient(s) that are not released will have no therapeutic effect. Second, the active ingredient(s) penetrate the skin tissue, an organ whose nature is to prevent this process.

Active ingredient released from a hydrophobic polymer system can be facilitated in at least two ways. The first is by addition of surfactants that will help the release thereof. By choosing certain surfactants, an increase in the partition of the active ingredient(s) from the polymer system can be enhanced when the polymer system includes a hydrophobic phase and a aqueous phase, thus enhancing the release of the active(s). The second method is to decrease the hydrophobic character of the polymer composition. The polymer compositions described herein are capable of adjustment to favor a lower level of hydrophobic character.

Once the active ingredient(s) are released from the transdermal delivery composition, the active ingredient(s) need to penetrate the skin tissue in order to be taken into the body. An issue regarding penetration of the skin tissue is the molecular weight of the active ingredient(s). In general, a molecular weight of less than about 600 Da would be expected to have the best chance of penetrating the skin tissue. It should be appreciated, however, that the active ingredient(s) can have a molecular weight of up to about 1000 Da and penetrate the skin tissue, but the ability to penetrate might be dependent on the active being sufficiently hydrophobic. In any case, it may be helpful that the active ingredient(s) have no charge and are slightly hydrophobic. Furthermore, compounds that are known as skin penetrants can help or assist with the active ingredient(s) being able to be taken up.

### Polymer Component

The polymer component that can be used in the composition can be a polymer adduct according to U.S. Pat. Nos. 8,318,818 and 8,481,058, the disclosures of which are incorporated herein by reference. The polymer adduct can be prepared by melt processing a hydrophobic polymer composition and a hydrophilic polymer composition to provide an interaction between the hydrophobic polymer composition and the hydrophilic polymer composition. It should be understood that the phrase “melt processing” refers to mixing the hydrophobic polymer composition and the hydrophilic polymer composition under conditions that provide that the hydrophobic polymer component of the hydrophobic polymer composition and the hydrophilic polymer component of the hydrophilic polymer composition are in a liquid state so that they sufficiently mix. When the polymers are sufficiently mixed, an interaction forms between the hydrophobic polymer component and the hydrophilic polymer component. The melt processing temperature is preferably at least about 90° C., more preferably at least about 100° C., and more preferably at least about 105° C. to generate this interaction.

The interaction exhibited between the hydrophobic polymer component and the hydrophilic polymer component can be considered a type of complex formation reaction, and that the complexes, once formed, are stable in water at temperatures up to 65° C. and at a pH range of 3.0 to 9.0. By stable, it is meant that the complexes do not favor disassociation. This interaction provides the composition with an ability to bind or hold onto the active ingredient(s) that are emulsified in water, and provides the composition with an ability to bind to skin and/or substrates of predominantly hydrophobic character.

### Hydrophobic Polymer Composition

The hydrophobic polymer composition that can be used includes repeating pyrrolidone/alkylene groups. Exemplary polymers that have repeating pyrrolidone/alkylene groups include those polymers obtained by a polymerizing alkylene substituted vinylpyrrolidone. The polymers can be represented by the following general formula:

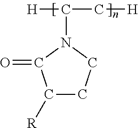

wherein R represents a carbon chain substitute such as an alkylene group and n represents the number of repeating units. The R group is preferably sufficiently long so that the polymer remains relatively water insoluble and should not be too long so that the polymer is difficult to melt process. Preferably, the alkylene group contains a length of at least about 10 carbon atoms and contains no more than about 25 carbon atoms. Preferably, the alkylene group contains between about 14 carbon atoms and about 22 carbon atoms, and more preferably between about 15 carbon atoms and about 19 carbon atoms.

The poly(vinylpyrrolidone/alkylene) polymers that can be used preferably have a molecular weight that is sufficiently high so that the polymer maintains its water insolubility but the molecular weight should not be so high that it becomes difficult to melt process the polymer. Preferably, the weight average molecular weight of the poly(vinylpyrrolidone/alkylene) polymer is between about 3,000 and about 400,000. Another way to characterize the size of the poly(vinylpyrrolidone/alkylene) polymer is by the number of repeating units (n). In the case of a poly(vinylpyrrolidone/alkylene) polymer having a weight average molecular weight of between about 6,000 and about 30,000, the poly(vinylpyrrolidone/alkylene) polymer has between about 20 and about 80 repeating units, and more preferably between about 30 and about 50 repeating units. It should be understood that repeating units refer to the residues of vinylpyrrolidone/alkylene groups.

Preferred poly(vinylpyrrolidone/alkylene) polymers that can be used include poly(vinylpyrrolidone/1-cicosene) and poly(vinylpyrrolidone/hexadecene). Poly(vinylpyrrolidone/1-eicosene) can be referred to as PVPE and is commonly used in pharmaceutical and cosmetic preparations. A preferred form of PVPE for use according to the invention includes about 43 to 44 repeating units in length and has a weight average molecular weight of about 17,000 and can be characterized as a paraffin-like solid. This particular PVPE is highly insoluble in water, and has an extremely low oral toxicity (LD₅₀>17000 mg/kg) and exhibits no demonstrable dermal toxicity. Poly(vinylpyrrolidone/1-hexadecene) can be referred to as PVPH. A preferred form of PVPH is available as a viscous yellow liquid that is insoluble in water and has a low oral toxicity (LD₅₀>64000 mg/kg), has about 39 to 40 repeating units, a molecular weight of about 12,000, and exhibits no demonstrable dermal toxicity.

PVPE and PVPH differ in the length of the hydrocarbon side chain, and are used extensively in the skin care industry, usually in concentrations of less than 1% by weight, because of their ability to bind to skin. Because the skin care industry generally prefers to apply actives to skin using a water-based composition, the use of PVPE and PVPH often requires solvents, surfactants, and emulsifiers to stabilize these polymers in a water emulsion. However, many of the solvents, surfactants and emulsifiers used to stabilize PVPE and PVPH in a water emulsion lack the low dermal toxicities of PVPE and PVPH. PVPE and PVPH by themselves lack a cosmetically elegant appeal when applied directly to the skin. They tend to be sticky and greasy.

The hydrophobic polymer composition is preferably provided as a mixture of different poly(vinylpyrrolidone/alkylene) polymers. The mixtures of different poly(vinylpyrrolidone/alkylene) polymers preferably include at least 5 wt. % of a first poly(vinylpyrrolidone/alkylene) polymer based on the weight of the hydrophobic polymer composition. The hydrophobic polymer composition preferably includes between about 5 wt. % and about 54 wt. % of the first poly(vinylpyrrolidone/alkylene) polymer. The second poly(vinylpyrrolidone/alkylene) polymer is preferably provided in an amount of at least about 46 wt. % and preferably in a range of between about 46 wt. % and 95 wt. %. For a hydrophobic polymer composition containing a first poly(vinylpyrrolidone/alkylene) polymer and a second poly(vinylpyrrolidone/alkylene) polymer, the mole ratio of the first polymer to the second polymer is preferably between about 1:22 and about 1:1. In general, when the hydrophobic polymer composition contains a mixture of different poly(vinylpyrrolidone/alkylene) polymers, it is preferable to provide at least one of the poly(vinylpyrrolidone/alkylene) polymers in an amount that provides improved properties to the composition compared to a composition having a hydrophobic polymer composition containing a single poly(vinylpyrrolidone/alkylene) polymer.

When the hydrophobic polymer composition is provided as a mixture of PVPH and PVPE, it is preferable that the PVPH is provided in the range of between about 46 wt. % to about 95 wt. % and the PVPE is provided in the range of between about 5 wt. % and about 65 wt. %, based upon the weight of the hydrophobic polymer composition.

### Hydrophilic Polymer Composition

The hydrophilic polymer composition that can be used includes at least one hydrophilic polymer and may include a mixture of hydrophilic polymers. The hydrophilic polymers that can be used include polymers having repeating carboxylic acid groups and/or hydroxyl groups. Preferred hydrophilic polymers that can be used according to the invention include polyacrylic acid polymers and poly(maleic acid/methylvinylether) copolymers.

Polyacrylic acid polymers that can be used preferably have a weight average molecular weight of at least about 50,000, and more preferably between about 50,000 and about 4,000,000. In addition, the polyacrylic acid polymers preferably have a level of cross-linking that is less than about 1%. A general structural representation of polyacrylic acid polymers is shown below:

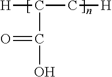

wherein n is the number of repeating units and is preferably between about 1,000 and about 20,000.

Poly(maleic acid/methylvinylether) copolymers that can be used preferably have a weight average molecular weight of at least about 50,000, and preferably between about 50,000 and about 4,000,000. The weight average molecular weight is more preferably between about 70,000 and 2,500,000. A general structural representation of poly(maleic acid/methylvinylether) copolymers is shown below:

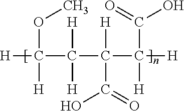

wherein n is the number of repeating units and is preferably between about 200 and about 20,000.

Additional hydrophilic polymers that can be used include starch, derivatives of starch, polyvinyl alcohol, cellulose, derivatives of cellulose, carboxymethyl cellulose, cyclodextrins, and dextrans. Exemplary starches include amylopectin and polyglucose. The weight average molecular weight of the hydrophilic polymers is preferably sufficient to provide solubility in water but not too high to become difficult to process. Starches that can be used according to the invention preferably have a weight average molecular weight of between about 50,000 and about 20,000,000. A derivative of starch that can be used includes partially hydrolized starch. Cellulose that can be used preferably has a weight average molecular weight of between about 50,000 and about 15,000,000. Polyglucose that can be used can be characterized as low fraction polyglucose having a weight average molecular weight of between about 60,000 and about 90,000, and high fraction polyglucose having a weight average molecular weight of between about 90,000 and about 300,000. An exemplary low fraction polyglucose material that can be used is available under the name Dextran-70. In general, this type of polyglucose has all alpha 1-6 linkages. Starch derivatives that can be used according to the invention include those starch derivatives having alpha 1-4 linkages. An example of this type of starch derivative includes cyclodextrins. Preferred cyclodextrins that can be used are those that act to provide a cavity within the molecule large enough to contain components desirable for applications. Preferably, the cyclodextrins that can be used have a molecular weight of between about 900 and about 1,400. Polyvinyl alcohols that can be used preferably have a weight average molecular weight of between about 50,000 and about 200,000.

Exemplary hydrophilic polymers that can be used include those polymers having the following melting temperature range and the following maximum temperature range beyond which it is expected decomposition of the polymer will occur. Exemplary poly(maleic acid/methylvinylether) copolymers that can be used include those having a melting temperature range of between about 60° C. and about 65° C. and a maximum temperature range of between about 80° C. and about 90° C. Exemplary polyacrylic acid polymers that can be used include those having a melting temperature range of between about 65° C. and about 70° C. and a maximum temperature range of between about 80° C. and about 90° C. Exemplary carboxymethyl cellulose polymers that can be used include those having a melting temperature range of between about 55° C. and about 60° C. and a maximum temperature range of between about 75° C. and about 80° C. Exemplary polyvinyl alcohol polymers that can be used include those having a melting temperature range of between about 50° C. and about 55° C. and a maximum temperature range of between about 65° C. and about 70° C. Exemplary starches that can be used include those having a melting temperature range of between about 40° C. and about 45° C. and a maximum temperature range of between about 50° C. and about 55° C. Exemplary dextrans that can be used include those having a melting temperature range of between about 37° C. and about 40° C. and a maximum temperature range of between about 45° C. and about 50° C. Exemplary β-cyclodextrins that can be used include those having a melting temperature range of between about 40° C. and about 45° C. and a maximum temperature range of between about 65° C. and about 70° C.

### Processing

The hydrophobic polymer composition and the hydrophilic polymer composition are preferably combined and mixed as a polymer melt. Preferably, the mixture is heated to at least about 75° C. to provide the polymer melt. Sufficient mixing is introduced to the polymer melt to form a complex between the hydrophobic and hydrophilic polymers. The composition is preferably heated to at least about 80° C., and more preferably at least about 90° C. under mixing to form a complex between the hydrophobic and hydrophilic polymers.

The complex formation step is preferably carried out in a relatively anhydrous environment. That is, the amount of water provided in the composition during the complex formation step is preferably less than about 1 wt. %. Once the desired level of complex formation has occurred, the composition can be hydrated with water.

The hydrophobic polymer composition and the hydrophilic polymer composition are preferably mixed together in amounts sufficient to provide a ratio of pyrrolidone groups to the combination of carboxylic acid groups and hydroxyl groups of between about 1:1 and about 5:1. The ratio of the structures causing the observed interaction between the hydrophobic polymer composition and the hydrophilic polymer composition can be referred to as “functional group parity.” Preferably, the ratio of pyrrolidone groups to the combination of carboxylic acid groups and hydroxyl groups is between about 1.5:1 and about 3:1. In order to drive the complex formation reaction, it is desirable to provide an imbalance between the two types of groups. Accordingly, it is generally desirable to provide more of the pyrrolidone groups than the combination of carboxylic groups and the hydroxyl groups. It should be understood that the reference to a “combination of carboxylic groups and hydroxyl groups” refers to the total amount of carboxylic groups and hydroxyl groups present but does not require the presence of both carboxylic groups and hydroxyl groups. For example, the value of the combination of carboxylic groups and hydroxyl groups can be determined for a composition that contains only carboxylic groups. Similarly, the value can be determined for a composition that contains only hydroxyl groups.

During the complex formation step, the amounts of hydrophobic polymer composition and hydrophilic polymer composition can be characterized on a weight percent basis. Preferably, about 2 wt. % to about 28 wt. % hydrophilic polymer composition and about 72 wt. % to about 98 wt. % hydrophobic polymer composition are combined to provide for complex formation. Preferably, about 8 wt. % to about 25 wt. % hydrophilic polymer composition and about 72 wt. % to about 95 wt. % hydrophobic polymer composition are combined to form the complex. During the complex formation step, the amount of water available in the composition is preferably less than about 1 wt. %. Although the complex forming composition can be relatively anhydrous, it is expected that the amount of water will be between about 0.3 wt. % and about 1.0 wt. %.

Once the hydrophobic polymers and the hydrophilic polymers have sufficiently reacted or interacted to form complexes, it is desirable to add water to the composition to provide a stable aqueous composition that can be relatively easily further hydrated. The stable aqueous composition that can be easily diluted further with water to form the use solution can be referred to as the concentrate. It is generally desirable to hydrate the composition to a water content that provides a relatively stable composition and that allows for water to be added at a later date without much difficulty. Although water can be added to the composition to a level equivalent to the level of the composition use solution, it is desirable to minimize the amount of water to avoid having to ship water. Shipping excess water is expected to add cost to the composition. In addition, it has been found that the first hydration of the composition precursor is the most difficult hydration step because of the need to control the conditions of hydration. After the first hydration to a water content of at least about 30 wt. %, it is expected that further hydrations to higher water contents are relatively easy and can be accomplished by simply mixing the composition with water. Accordingly, the amount of water provided in the composition when made available as a concentrate for shipment is preferably between about 30 wt. % and about 45 wt. %. When the composition includes about 30 wt. % to about 45 wt. % water, it is expected that the composition will include between about 3 wt. % and about 10 wt. % hydrophilic polymer composition and between about 30 wt. % and about 50 wt. % hydrophobic polymer composition.

Water is added to the relatively anhydrous composition by mixing water and the relatively anhydrous composition at a temperature and for a time sufficient to allow the composition to become hydrated without losing significant amounts of interaction between the hydrophobic polymer composition and the hydrophilic polymer composition. In general, the relatively anhydrous composition is hydrated by heating to at least 60° C. and adding water while mixing. Preferably, the composition is heated to at least about 65° C. and more preferably at least about 70° C. A preferred temperature range is about 65° C. to about 80° C.

The relatively anhydrous composition can be referred to as the composition concentrate. The composition having a water concentration of between about 30 wt. % and about 95 wt. % can be referred to as the concentrate. The manufacturers of the composition may further hydrate the composition or use it as it is. The transdermal delivery composition can include a sufficient amount of the adduct to hold the active ingredient(s) in the composition while also releasing the active ingredient(s) from the composition over a time period of about 4 to 12 hours, preferably about 4 to 6 hours, and also provide a consistent and sustained release. Preferably, the transdermal delivery composition contains about 4 wt. % to about 9 wt. %, more preferably about 4.5 wt. % to about 8 wt. %, and even more preferably about 5 wt. % to about 6 wt. % of the adduct.

For enhancing the release of active ingredient(s) from the composition, it may be desirable to reduce or lower the hydrophobic character of the polymer complex. This can be done, for example, by providing the polymer complex with about 3 to about 10% polyvinylpyrrolidone hexadecene and about 3 to about 7% of polyvinylpyrrolidone eicosene.

### Surfactants

Surfactants can be incorporated into the transdermal delivery composition to provide help solubilize the polymer adduct and to help control the release of active ingredient or other components. It is expected that the amount of surfactant and the type of surfactant can be adjusted as desired. In the case where an active ingredient or other component desired to be released is relatively more hydrophobic, it is expected that by increasing the surfactant concentration, an equilibrium shift favors the aqueous phase and promotes a faster release of the ingredient. Surfactants can be useful for releasing active ingredient(s) from the transdermal delivery composition because the active ingredient(s) can be characterized as hydrophobic.

Surfactants that can be incorporated into the composition according to the invention include nonionic surfactants, cationic surfactants, anionic surfactants, and amphoteric surfactants, and mixtures thereof. It may be desirable to use surfactants when they have a tendency to increase the emulsion stability and/or to promote the release of active ingredients. That is, surfactants can be used to increase the water solubility of the polymers of the composition and/or the surfactants can be used to decrease the binding of the active ingredient(s) to the polymers to thereby enhance release of the active ingredient(s).

Nonionic surfactants that can be used include ethoxylated, propoxylated, ethoxylated-propoxylated surfactants, and mixtures thereof. An exemplary nonionic surfactant that can be use includes nonylphenol ethoxylate having nine ethylene oxide groups and is available under the name Nonoxynol-9. Additional exemplary nonionic surfactants that can be used include Poloxamer surfactants such as Poloxamer 124 and Poloxamer 237, and are available under the trade names Synperonics, Pluronics, and Kolliphor.

Anionic surfactants that can be used include carboxylic salts (soaps) and sulfonate salts (detergents). Cationic surfactants that can be used include amides such as cocoamide. One concern with the use of anionic surfactants, cationic surfactants, and amphoteric surfactants relates to the potential destabilization of emulsions as a result of the presence of salts. Accordingly, it may be desirable to use anionic surfactants, cationic surfactants, and amphoteric surfactants at sufficiently low levels to reduce this destabilizing effect. It is expected that these surfactants will be used at lower levels than nonionic surfactants. In addition, the positive charge of the cationic surfactants and the amphoteric surfactants can have an effect of forming insoluble complexes with portions of the hydrophilic polymer composition.

When surfactants are used, it is generally desirable to use the surfactant or mixture of surfactants in an amount that provides a desired level of emulsion stability and provides a desired rate of release of active ingredient(s). It is expected that in most applications, the surfactant or mixture of surfactants will be provided at a concentration of about 3 wt. % to about 9 wt. %, preferably about 3.5 wt. % to about 8 wt. %, and more preferably about 4 wt. % to about 6 wt. %.

### Skin Penetrants

Skin penetrants can be referred to as skin penetrators, penetrant components, or penentrants, and can be incorporated into the transdermal delivery composition to enhance penetration of the active ingredient(s) through the skin tissue. Once the active ingredient(s) have been released from the composition, the penetrant helps the active ingredient(s) enter through the skin tissue. Exemplary skin penetrants that can be used include caprylic acid, capric acid, lauric acid, myristic acid, palmitic acid, linoleic acid, linolenic acid, oleic acid, elaidic acid, 10-methyl palmitic acid, 10-methyl hexadec-9-enoic acid, 7-methyl octanoic acid, 9-methyl decanoic acid, 8-ethyl decanoic acid, 10-ethyl dodecanoic acid, lauryl choline, terpenes, anethole, α-bisabolol, sesquiterpene, borneol, camphor, carvacrol, carvone, 1,8-cineole, cymene, 1,4-cineole, cymene, eugenol, linalool, menthol, menthone, menthol menthone, farnesol, geraniol, limonene, nerolidol, α-pinene, neorxoildideol, α-pinene oxide, α-pinene oxide pulegone, terpinen-4-ol (4-te(r4p-tienrepninoeln)ol), tetra-hydrogeraniol, thymol valen-cene, verbenon-e, and mixtures thereof. Preferred skin penetrants include a mixture of oleic acid and terpene.

The transdermal delivery composition can include a penetrant in an amount sufficient to enhance the transdermal delivery of the active ingredient(s). Preferably, the transdermal delivery composition contains at least about 1 wt. % penetrant, more preferably about 2 wt. % to about 10 wt. % penetrant, more preferably about 3 wt. % to about 7 wt. % penetrant, and more preferably about 3.5 wt. % to about 5 wt. % penetrant.

### Antioxidants

The transdermal delivery composition can include antioxidants to help reduce decomposition of the active ingredient(s). Exemplary antioxidants that can be used include β-carotene, lycopene, lutein, glutathione, melatonin, oestrogen, ubiquinol-10, N-acetyl cysteine, lipoic acid (LA), zinc, selenium, copper, quercetin, catechin, cortisone, estradiol, estriol, α-tocopherol, ascorbic acid, butylatedhydroxytoluene (BHT), butylatedhydroxyanisole (BHA), 2,3,-dimercaptosuccinic acid (DMSA), monoisoamyldimercaptosuccinic acid, dihydrolipoic acid (DHLA), gallic acid, propygallate, α-tocopherolacetate, vitamin E, vitamin E acetate, synthetic forms of vitamin E, and mixtures thereof. The antioxidant is an optional component, but when the transdermal delivery composition contains an antioxidant, it can include less than about 0.03 wt. % antioxidant, and alternatively, preferably about 0.1 wt. to about 1 wt. % antioxidant, and more preferably about 0.2 wt. % to about 0.5 wt. % antioxidant.

### Chelating Agents

The transdermal delivery composition can include a chelating agent to react with metal ions in the water of the composition to form a stable, water-soluable complex. Various exemplary chelating agents are well known. A preferred chelating agent can be used includes ethylenediamientetraacetic acid (EDTA). The presence of a chelating agent in the composition is optional and typically depends on the quality of the water used to form the composition. When included in the transdermal delivery composition, the chelating agent is preferably provided in an amount sufficient to provide chelating properties. Exemplary ranges of chelating agent that can be included in the transdermal delivery composition includes less than about 0.05 wt. %, and alternatively preferably about 0.01 wt. % to about 0.1 wt. %.

### Emollients

The transdermal delivery composition can include an emollient to provide a desirable feel to the composition. Exemplary emollients that can be used include mineral oil, lanolin oil, coconut oil, cocoa butter, olive oil, almond oil, macadamia nut oil, synthetic jojoba oils, natural sonora jojoba oils, safflower oil, corn oil, aloe vera, cottonseed oil, peanut oil, squalene, castor oil, polybutene, odorless mineral spirits, sweet almond oil, avocado oil, clophyllum oil, ricin oil, vitamin E acetate, linolenic alcohol, oleyl alcohol, cereal germ oils, wheat germ oil, isopropyl palmitate, isopropyl myristate, hexadecyl stearate, butyl stearate, decyl oleate, acetyl glycerides, octanoates and benzoates of (C₁₂-C₁₅) alcohols, octanoates and decanoates of alcohols and polyalcohols glycol and glycerol, isopropyl adipate, hexyl laurate, octyl dodecanoate, hydrogenated lanolin, hydroxylated lanolin, acetylated lanolin, petrolatum, isopropyl lanolate, butyl myristate, cetyl myristate, myristyl myrislate, myristyl lactate, cetyl alcohol, isostearyl alcohol, isocetyl lanolate, stearic acid, stearyl alcohol, palmitic acid esters, natural and synthetic esters such as coconut oil, and mixtures thereof.

The presence of an emollient in the composition is optional, but when it is present, it is preferably present in an amount of about 1 wt. % to about 5 wt. %, and more preferably about 1.5 wt. % to about 4 wt. %.

### Water

The transdermal delivery composition can include an amount of water so that the composition can be applied conveniently to the skin tissue. This amount of water incudes the amount of water in the other components. Preferably, the transdermal delivery composition includes water in an amount of about 70 wt. % to about 90 wt. %, and preferably about 75 wt. % to about 85 wt. %.

### PH Neutralizing Agent

The transdermal delivery composition is preferably provided at a pH sufficient for the delivery and penetration of the active ingredient(s). The composition can include a pH neutralizing agent to provide the composition with a pH in the range of about 6 to about 7.5. Known PH neutralizing agents can be used including, for example, triethanolamine (TEA).

### Additional Components

The transdermal delivery composition is preferably prepared by mixing the components for the formation of the transdermal delivery composition. Components that can be incorporated into the composition for forming the use solution include those components normally encountered in the topical composition industry. Exemplary components include preservatives such as antimicrobial agents. The presence of a preservative in the composition is optional, but when it is present, it is preferably present in an amount of about 0.1 wt. % to about 2 wt. %, and more preferably about 0.5 wt. % to about 1.5 wt. %.

### Application

The transdermal delivery composition can be applied to skin tissue in an amount that provides a desired effect. The composition can be applied in a volumetric amount desired to clean, dry skin. Preferably to the solar forearm or an area of concern, spread to an even film, and allowed to dry. The effects can generally be felt or noticed within about 15 to 20 minutes. The release of the active ingredient(s) from the composition can occur after or upon application of the composition to the skin tissue, and the release of both the active ingredient(s) and the penetrant allows the penetrant to help or assist with the passage of the active ingredient(s) through the skin tissue so that the active ingredient(s) can be taken into the system and act at the appropriate locations to provide the desired effects. Also, it may be advantageous that the active ingredient(s), as a result of the transdermal delivery, may provide desired efficacy without being processed through, for example, the liver, thereby providing quicker action and with a lower dosage compared with other delivery techniques.

The transdermal delivery system described herein may be a safe and effective delivery method for CB-1 receptor antagonists and/or GLP-1 receptor agonists and/or SGLT-2 receptor antagonist.

### EXAMPLES

The following examples were carried out to demonstrate advantages of the present disclosure. It should be understood that the invention is not limited to the examples of this application.

### Example 1

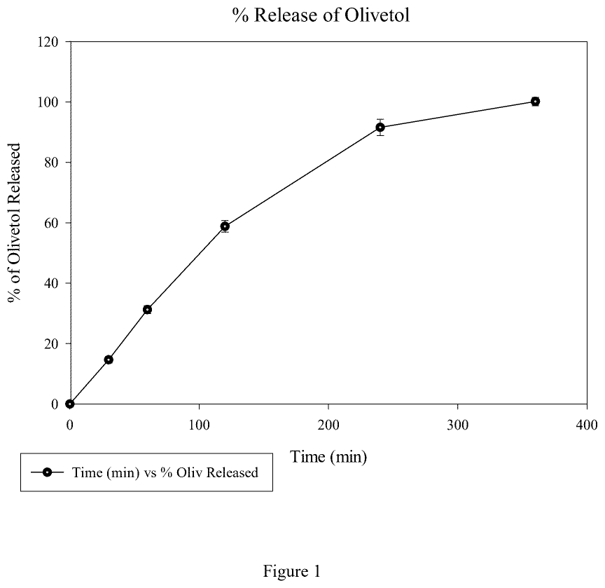

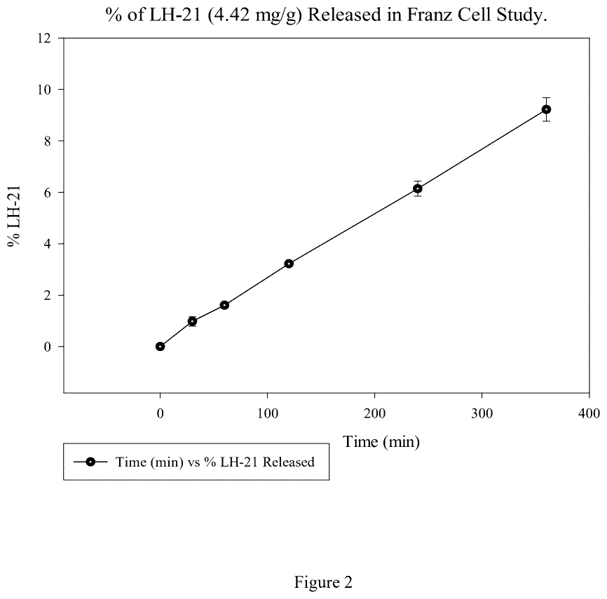

Franz cell studies were conducted on two products with the delivery system described above, and Olivetol at a dose of 6.77 mg/g or LH-21 at a dose of 4.42 mg/g. Results of the Franz cell studies are shown in FIGS. 1 and 2. FIG. 1 shows the release and membrane penetration of Olivetol at a dose of 6.77 mg/g, and FIG. 2 shows the release and membrane penetration of LH-21 at a dose of 4.42 mg/g.

### Example 2

Danuglipron was purchased from MedChemExpress. A transdermal product containing 0.44% Danuglipron was manufactured according to Table 1. The product was placed on an accelerated stability test at 400 and 25° C.

1.0 to 1.5 mls of product were introduced into the donor cups of 3 Franz Cells. Receiver chambers were filled to capacity (˜8.0 ml) with phosphate buffered saline at a pH of 7.0.

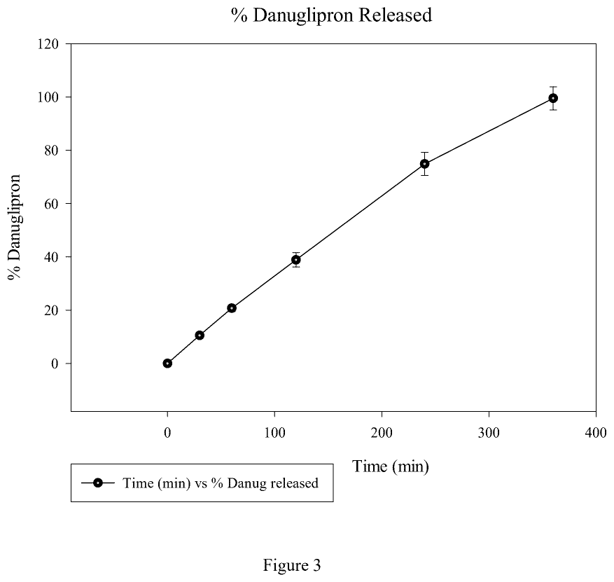

Samples of 250 mcrL were removed from the receiver chambers at 30 to 360 minutes. Data of % Danuglipron released, mg released, and mg/cm² are shown in FIG. 3.

### Example 3

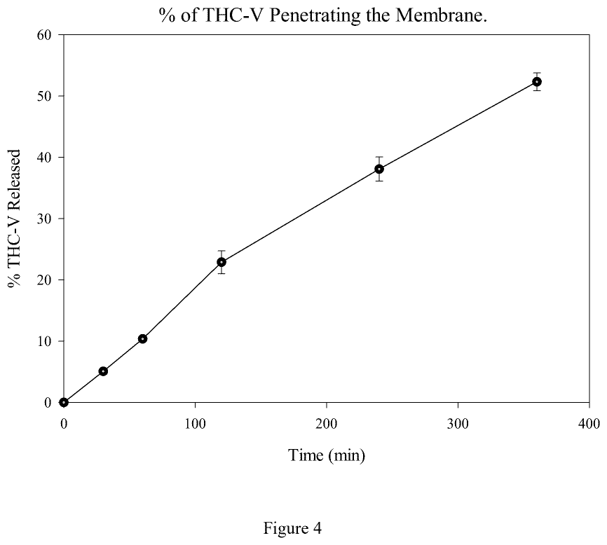

A transdermal formulation of 1% THCV was prepared according to Table 1. The THCV formulation was subjected to a Franz Cell study, measuring the rate of diffusion across a cellulose membrane. Results are shown in FIG. 4. As illustrated by the results, after 6 hours, 6.08 mg of THCV diffused across the cellulose membrane. The total dose of THCV applied to the membrane was 11.63 mg, and this calculates to a delivered dose of 52.3% of the THCV which is unexpected.

Based on the examples, it can be seen that the transdermal delivery composition can provide a sustained release and permeation of the at least one active over a period of at least four hours, and preferably six hours, wherein the release in each hour after the first hour can be a release and permeation of the active that is between about 50% and 200% of the release and permeation of the active in a preceding hour and is based on a Franz Cell Study. Preferably, the release and permeation of the active is between about 75% and 150% of the release and permeation of the active in a preceding hour.

The above specification, examples and data provide a complete description of the manufacture and use of the composition of the invention. Since many embodiments of the invention can be made without departing from the spirit and scope of the invention, the invention resides in the claims hereinafter appended.

1. A transdermal delivery composition comprising:
(a) a therapeutic effective amount of at least one glucose controlling agent;
(b) a polymer component in an amount sufficient to hold the at least one glucose controlling agent and provide a sustained release of the at least one glucose controlling agent from the transdermal composition after application to skin tissue;
(c) a penetrant component in an amount sufficient to assist with a transdermal penetration of the at least one glucose controlling agent;
(d) a surfactant component in an amount sufficient to stabilize the polymer adduct and release the at least one glucose controlling agent from the transdermal delivery composition after application to skin tissue; and
(e) water.

2. A transdermal delivery composition according to claim 1, wherein the at least one glucose controlling agent comprises at least one CB-1 receptor antagonist, at least one GLP-1 receptor agonist, at least one SGLT-2 receptor antagonist, or a combination thereof.

3. A transdermal delivery composition according to claim 1, wherein the polymer component comprises at least one hydrophilic polymer/hydrophobic polymer adduct.

4. A transdermal delivery composition according to claim 3, wherein the hydrophobic polymer/hydrophilic polymer adduct comprises at least two different poly(vinylpyrrolidone/alkylene) polymers, wherein the alkylene group of each polymer contains at least 10 carbon atoms.

5-8. (canceled)

9. A transdermal delivery composition according to claim 1, wherein the at least one glucose controlling agent has a molecular weight of about 1,000 Da or less.

10. (canceled)

11. (canceled)

12. A transdermal delivery composition according to claim 1, wherein the at least one glucose controlling agent comprises a CB-1 receptor antagonist, and the CB-1 receptor antagonist comprises at least one of olivetol, LH-21, rimonavant, taranabant, Monlunabant, INV-347, and INV-202 (CagriSema), tetrahydrocannbivarin, or mixtures thereof.

13. (canceled)

14. A transdermal delivery composition according to claim 1, wherein the at least one glucose controlling agent comprises a GLP-1 receptor agonist, and the GLP-1 receptor agonist comprises exenatide, liraglutide, Orforglipron, Lotiglipron, Danuglipron, GT-001, GDD-3898, GSBR-1290, RG 6652 (CT-996), or mixtures thereof, or wherein the at least one glucose controlling agent comprises a dual GLP-1/GIP receptor agonist, and the dual GLP-1/GIP receptor agonist comprises RG6641 (CT-868) and RG 6640 (CT-388), or mixtures thereof.

15-17. (canceled)

18. A transdermal delivery composition according to claim 1, wherein the at least one glucose controlling agent comprises a SGLT-2 receptor antagonist, and the SGLT-2 receptor antagonist comprises canagliflozin (Invokana), ertugliflozin (Steglatro), dapagliflozin (Farxiga), and empagliflozin (Jardiance), or mixtures thereof.

19. A transdermal delivery composition according to claim 2, wherein the composition comprises the at least one CB-1 receptor antagonist in an amount of about 0.05 wt. % to about 4 wt. %, the at least one GLP-1 receptor agonist in an amount of about 0.05 wt. % to about 4 wt. %, the at least on SGLT-2 receptor antagonist in an amount of about 0.05 wt. % to about 4 wt. %, or the combination thereof in an amount of at least 0.05 wt. % to about 4 wt. % based on the weight of the composition.

20-29. (canceled)

30. A transdermal delivery composition according to claim 1, wherein the composition comprises the penetrant component in an amount of about 2 wt. % to about 10 wt. % based on the weight of the composition and the surfactant component in an amount of about 3 wt. % to about 9 wt. % based on the weight of the composition.

31-33. (canceled)

34. A transdermal delivery composition according to claim 1, wherein the composition comprises at least 70 wt. % of the water based on the weight of the composition.

35. (canceled)

36. A transdermal delivery composition according to claim 1, wherein the transdermal delivery composition provides a sustained release and permeation of the at least one glucose controlling agent over a period of at least four hours wherein the release in each hour after the first hour can be a release and permeation of the at least one glucose controlling agent that is between about 50% and 200% of the release and permeation of the at least one glucose controlling agent in a preceding hour and is based on a Franz Cell Study.

37. (canceled)

38. A transdermal delivery composition comprising:
(a) a therapeutic effective amount of a CB-1 receptor antagonist comprising at least one of olivetol, LH-21, rimonabant, taranabant, tetrahydrocannbivarin, or mixtures thereof;
(b) a hydrophilic polymer/hydrophobic polymer adduct in an amount sufficient to hold the CB-1 receptor antagonist and provide a sustained release of the CB-1 receptor antagonist after application to skin tissue;
(c) a penetrant component in an amount sufficient to assist with a transdermal penetration of the CB-1 receptor antagonist;
(d) a surfactant component in an amount sufficient to stabilize the polymer adduct and release the CB-1 receptor antagonists from the transdermal delivery composition after application to skin tissue; and
(e) water.

39-45. (canceled)

46. A transdermal delivery composition according to claim 38, wherein the composition comprises the CB-1 receptor antagonist in an amount of about 0.05 wt. % to about 4 wt. % based on the weight of the composition.

47. (canceled)

48. A transdermal delivery composition according to claim 38, wherein the composition comprises at least two of the CB-1 receptor antagonists.

49-57. (canceled)

58. A transdermal delivery composition comprising:
(a) a therapeutic effective amount of a GLP-1 receptor agonist comprising at least one of exenatide, liraglutide, Orforglipron, Lotiglipron, Danuglipron, GT-001, GDD-3898, GSBR-1290, RG 6652 (CT-996), or mixtures thereof;
(b) a hydrophilic polymer/hydrophobic polymer adduct in an amount sufficient to hold the GLP-1 receptor agonist and provide a sustained release of the GLP-1 receptor agonist after application to skin tissue;
(c) a penetrant component in an amount sufficient to assist with a transdermal penetration of the GLP-1 receptor agonist;
(d) a surfactant component in an amount sufficient to stabilize the polymer adduct and release the GLP-1 receptor agonists from the transdermal delivery composition after application to skin tissue; and
(e) water.

59-64. (canceled)

65. A transdermal delivery composition according to claim 58, wherein the composition comprises the GLP-1 receptor agonist in an amount of about 0.05 wt. % to about 4 wt. % based on the weight of the composition.

66-76. (canceled)

77. A method for delivery of at least one glucose controlling agent through skin tissue, the method comprising:
applying a transdermal delivery composition according to claim 1 to the skin tissue and spreading the composition to form a film on the skin tissue.

78. A method according to claim 77, wherein the transdermal delivery composition provides a sustained release and permeation of the at least one glucose controlling agent over a period of at least four hours wherein the release in each hour after the first hour can be a release and permeation of the at least one glucose controlling agent that is between about 50% and 200% of the release and permeation of the at least one glucose controlling agent in a preceding hour and is based on a Franz Cell Study.

79-84. (canceled)

85. A method according to claim 77, wherein the at least one glucose controlling agent comprises at least one CB-1 receptor antagonist, at least one GLP-1 receptor agonist, at least one SGLT-2 receptor antagonist, or a combination thereof.
